(19) United States
(12) Patent Application Publication
(10) Pub. No.: US 2005/0192555 A1
(43) Pub. Date: Sep. 1, 2005

(54) Absorbent undergarments for obese body types

(76) Inventor: Camille M. Thomas, SeaTac, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/048,617

(22) Filed: Jan. 31, 2005

Related U.S. Application Data
(60) Provisional application No. 60/539,719, filed on Jan. 29, 2004.

(51) Int. Cl. A61F 13/15

(52) U.S. Cl. 604/402

(57) ABSTRACT

An incontinence undergarment and accessory for persons of obese body type. The accessory includes a belt member sized to surround the user's torso having an anterior and posterior segment joined by to connecting segments, and further includes at least one anterior and one posterior vertical suspension member, both having sufficient length to terminate proximate to or above the user's pelvis. The vertical suspension members provide a desired fixation location for receiving an absorbent incontinence component. The incontinence undergarment includes an absorbent panel having anterior and posterior vertically extending suspension members, which are linked to a belt member sized to encircle a user's torso. Variations of the invention include alternative belt member configurations for user's of different body mass and shape, and alternative vertical suspension member arrangements.

### BACKGROUND OF THE INVENTION

Current studies show that 30% of the US population is considered overweight by medical standards, and that 50% of the future US population will be considered obese. Of this population, a certain portion has or will have need for incontinence-type undergarments. Current solutions for this need primarily consist of providing “oversized” versions of conventional diapers or incontinence undergarments, that are sized based upon a user's waist or hip measurements. However, this solution does not take into account at least one very important factor.

A human body having an obese characterization as compared to an average body is not merely a scaled up version of the average body. Instead, obese body types are characterized as having disproportionate mass distributions about the body, which account for most of the mass differential. Primarily, the increased mass distribution occurs from below the breast to the top of the thighs, with a concentration of body fat around the central part of the body.

Because the body mass distribution for obese persons differs from that of non-obese persons, and because conventional “oversized” diapers or incontinence undergarments do not have the structure necessary to properly fit obese persons, the fit of such conventional diapers or incontinence undergarments is wanting. Moreover, all obese persons do not share the same type of mass distributions. Some persons have an “apple” mass distribution while others have a “pear” mass distribution. Consequently, any successful diaper or incontinence undergarment should take these diverging body types into consideration if a proper fit is desired.

### SUMMARY OF THE INVENTION

The invention is directed to an incontinence undergarment and system for persons of obese body types. The invention comprises an absorbent panel preferably having a first fluid absorption value, and support structure, preferably having a second absorption value less than the first absorption value, for maintaining the relative position of the absorbent panel with respect to the user's body during use of the undergarment. The support structure comprises a belt member for encircling the user's torso, an anterior suspension member extending from the belt member and a posterior suspension member extending from the belt member. Either or both of the suspension members may comprise all, part or none of the belt member, depending upon the mode of implementation. Each suspension member includes at least one vertical strap extending from the belt member and linking with the absorbent panel. Thusly configured and during use, the belt member provides an anchor for suspending the absorbent panel, through the anterior and posterior suspension members.

A feature of the invention provides for the accommodation of various body types associated with obese persons. Because obese persons frequently have an enlarged midsection at or above the pelvis region, conventional absorbent undergarments usually fail to maintain a proper position on the person's body. It is therefore considered desirable to provide a secure anchor location such that undesirable dropping of the undergarment can be prevented. By positioning the belt member above the enlarged mid section, a secure anchor location can be established. Through the use of suspension members extending from the belt member and having an appropriate length, the absorbent panel can be securely held in place.

To this end, appropriate length sizing of the suspension members can be accomplished by several non-exclusive means: a) providing a plurality of generally inelastic, pre-established length suspension members for each belt member having absorbent panel engagement means at the distal ends thereof (the engagement means may be either permanent or temporary); b) providing a plurality of generally elastic, pre-established length suspension members for each belt member having absorbent panel engagement means at the distal ends thereof (the engagement means may be either permanent or temporary); c) providing a plurality of generally inelastic, pre-established length suspension members for each belt member having absorbent panel engagement means at the distal ends thereof and at one or more locations between the distal ends and proximal ends (the engagement means may be either permanent or temporary); d) providing a plurality of generally elastic, pre-established length suspension members for each belt member having absorbent panel engagement means at the distal ends thereof and at one or more locations between the distal ends and proximal ends (the engagement means may be either permanent or temporary).

Another feature of the invention provides for lateral linkage between an anterior portion of the absorbent panel and a posterior portion of the absorbent panel. In a preferred embodiment, the lateral linkage is accomplished by at least one band or strap extending from the anterior portion of the absorbent panel to the posterior portion of the absorbent panel wherein the band or strap is attached (either removably with respect to both panels or just one) to the panels. Most preferably, at least two straps extend from lateral edge portions of one of the anterior or posterior portion of the absorbent panel to an opposing portion.

### BRIEF DESCRIPTION OF THE DRAWINGS

FIG. 1 is a perspective view of a first embodiment of the invention shown in a pre-use state, particularly illustrating multi-attachment point suspension members to permit user-selected distances between a belt member and an absorbent panel;

FIG. 2 shows a variation of the embodiment of FIG. 1 during use by an obese person having an “apple” body type;

FIG. 3 shows a variation of the embodiment of FIG. 1 during use by an obese person having a “pear” body type;

FIG. 4 is plan view of a first belt member arrangement;

FIG. 5 is plan view of a second belt member arrangement, particularly for use by very obese persons;

FIG. 6 is plan view of a third belt member arrangement;

FIG. 7 is a perspective view of a second embodiment of the invention shown in a pre-use state, particularly illustrating a lateral connection member arrangement and alternative multi-attachment point suspension members to permit user-selected distances between a belt member and an absorbent panel;

FIG. 8 is a side elevation view of the embodiment of FIG. 7 during use; and

FIG. 9 is a perspective view of a third embodiment of the invention wherein the anterior suspension member comprises a unitary vertical suspension member.

### DETAILED DESCRIPTION OF PREFERRED EMBODIMENTS

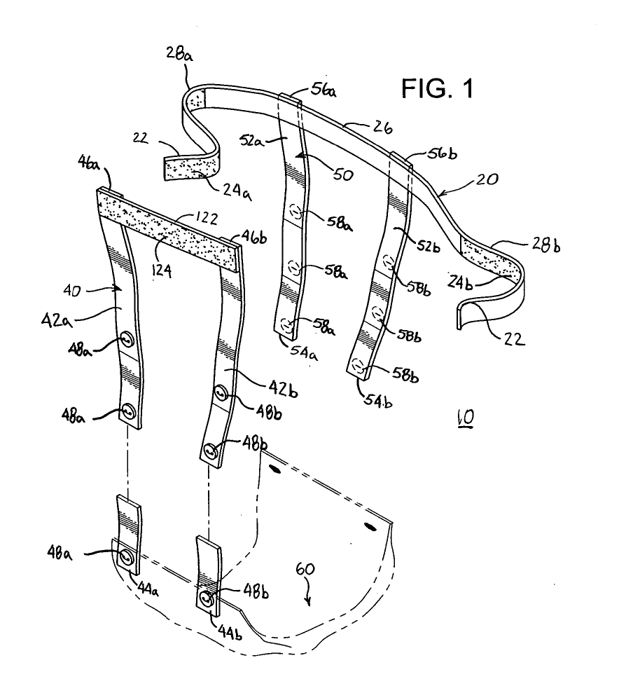

Turning then to the several Figures, wherein like numbers indicate like parts, and more particularly, to FIGS. 1-6, a first embodiment of the invention is shown. As best illustrated in FIG. 1, undergarment 10 comprises belt member 20, anterior suspension member 40, posterior suspension member 50, and absorbent panel 60. Those persons skilled in the art will appreciate the substitution of various materials for others. Thus, it is presently contemplated that absorbent panel 60 functions as a fluid retaining undergarment, the same as or similar to conventional infant diapers or adult incontinence pads. The basic construction of absorbent panel 60 prevents the unintentional escapement of fluids from the interior portion thereof to the environment, and further functions to bind such fluids in a manner to prevent unintentional continued contact with the user. In contrast, belt 20, anterior suspension member 40 and posterior suspension member 50 may be constructed of higher strength and lesser absorptive value material. Preferably, the belt 10 and suspension members 40 and 50 have a soft surface that contacts the user, while having an outer surface constructed from a material which beneficially provides the desirable high strength features.

Turning in particular to belt member 20, it comprises anterior belt portion 22, posterior belt portion 26, and lateral belt portions 28a/b. As used herein, the term “portions” may, depending upon the embodiment being discussed, refer to discrete segments or pieces of belt member 20, or may reference areas or zones of the belt member, with those persons being skilled in the art appreciating the diversity of construction available for a basic body encircling device. Thus, and as will be shown in additional embodiments, belt member 20 may be comprised of separate pieces or segments to be joined during use of undergarment 10, or may be a substantially continuous singular element, such as illustrated in FIG. 5, having identifiable areas or zones for labeling purposes.

Belt member 20 further comprises two-part fasteners 24a and 24b, which are positioned on the free ends of anterior belt portion 22. While an arrangement similar to that of a conventional belt may be used (wherein the inside surface of belt 20 has one part of a two-part fastener and the outside surface of belt 20 has a second part of a two-part fastener so that, when the ends of the anterior portion are overlapped, an encircling structure can be formed), the presently contemplated arrangement positions both fasteners on the same surface (a common surface) of anterior belt portion 22, as shown in FIG. 1 for the reasons expressed below. Fasteners 24a and 24b may be both first parts, both second parts, or respective first and second parts of the two-part fastener, depending upon design considerations. It is considered desirable to have at least one such fastener at or proximate to each free end of anterior belt portion 22, although such fastener may extend posteriorly or additional fasteners may be present posterior of the preferred location to increase fit or resistance to separation. As used herein, two-part fasteners include any means for removably fastening two flexible, panel-like materials together, such as chemical means, e.g., adhesives, cohesives; physical means, e.g., buttons and holes, laces; mechanically interacting means, e.g., hook and loop fasteners, slide fasteners.

Functioning in cooperation with belt member 20 in FIG. 1 is anterior suspension portion 122, which also unifies vertical straps 42a and 42b at first ends 46a and 46b. As noted above, fasteners 24a and 24b are preferably positioned on a common surface of anterior belt portion 22 at or near the free ends thereof. For ease of use, the most preferred location for fasteners 24a and 24b is on the inside surface of anterior belt-portion 22. The complementary portion to fasteners 24a and 24b can be found on the outer surface of anterior suspension portion 122 and are represented as fastener 124. This fastener, which may be segmented, is intended to be complementary to fasteners 24a and 24b found on anterior belt portion 22 of belt member 20. While a suitable functionality could be achieved by merely mating the free distal ends of anterior belt portion 22, a larger variety of user torso diameters is possible through the use of anterior suspension portion 122, and the corresponding second part of the two-part fasteners 124. The resulting arrangement permits encircling a user's torso through a wide range of diameters, as represented by the relative distance between the distal ends of anterior belt portion 122.

Both anterior suspension member 40 and posterior suspension member 50 are either removably or fixedly attached to absorbent panel 60 at respective attachment points 48a/b and 58a/b. The means of connection is considered to be wholly a design consideration, and those persons skilled in the art will appreciate the diversity of available attachment means, depending upon the construction of materials used and the intended purposes of the absorbent panel and supporting structure. Nevertheless, if an entirely disposable unit is desired, then use of stitching, RF spot welding, or adhesives are considered most economical, while reusable arrangements are considered more advantageous and may comprise use of hook-and-loop fasteners (two-part fastening systems) or more conventional forms, such as button and button holes, snap connectors and the like. Similarly, the attachment means for connecting vertical straps 42a/b and 52a/b to belt member 20 (and/or any portions or segments thereof) may be either removable or permanent. However, the embodiments shown herein presume that such attachment is permanent since this portion of undergarment 10 is clearly reusable.

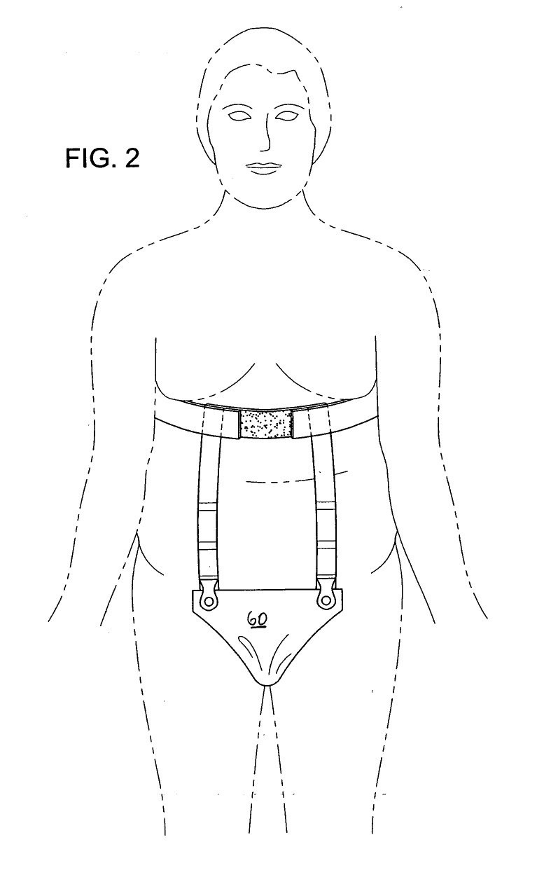

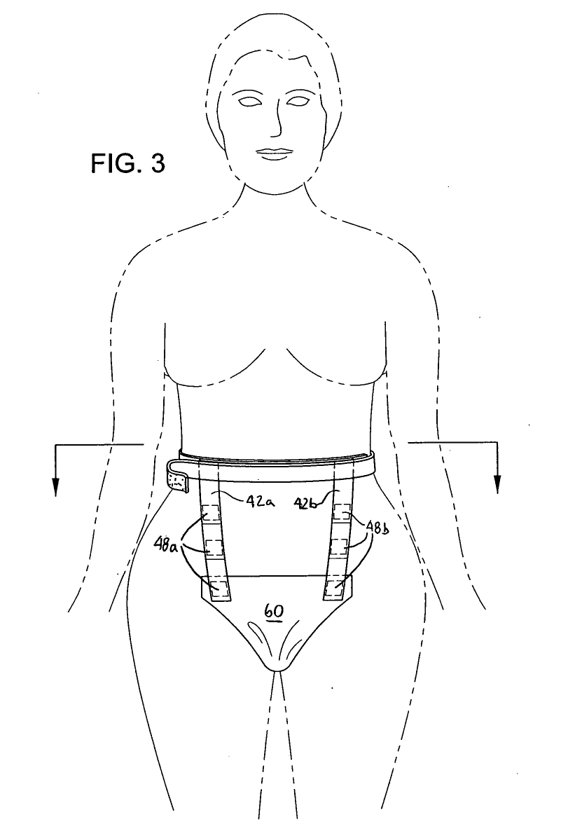

Turning momentarily to FIGS. 2 and 3, it can be seen that various users will have different positioning requirements. As a consequence of body features and mass distribution, certain users will find it necessary to place the belt member 20 above the waist, while other will find greater utility by placement of the belt member 20 proximate to the waist. To facilitate these differing modes of implementation, anterior and posterior suspension members 40 and 50, respectively, are preferably adjustable in length. This feature can be accomplished either through the use of resilient or elastic material, thereby permitting tension stretching of the suspension members; otherwise, temporarily adjustable vertical suspension members may be employed. In this regard, a plurality of attachment points 48a and 48b of vertical straps 42a and 42b and, similarly, a plurality of attachment points 58a and 58b of vertical straps 52a and 52b can be used.

Also shown in these Figures are several modes of securing belt member 20 to absorbent panel 60. In FIG. 2, a snap-type fastener (similar to that used with respect to bed sheets and certain garters) is shown, with attachment points 48a and 48b on vertical straps 42a and 42b being user selectable. In FIG. 3, adhesive strips covered by release film are located at attachment points 48a and 48b on vertical straps 42a and 42b; the user need only remove the release film to expose the adhesive strip of interest and firmly engage the same with absorbent panel 60. Again, it should be understood by those persons skilled in the art that the previously mentioned attachment means can be used in various combinations depending upon design considerations.

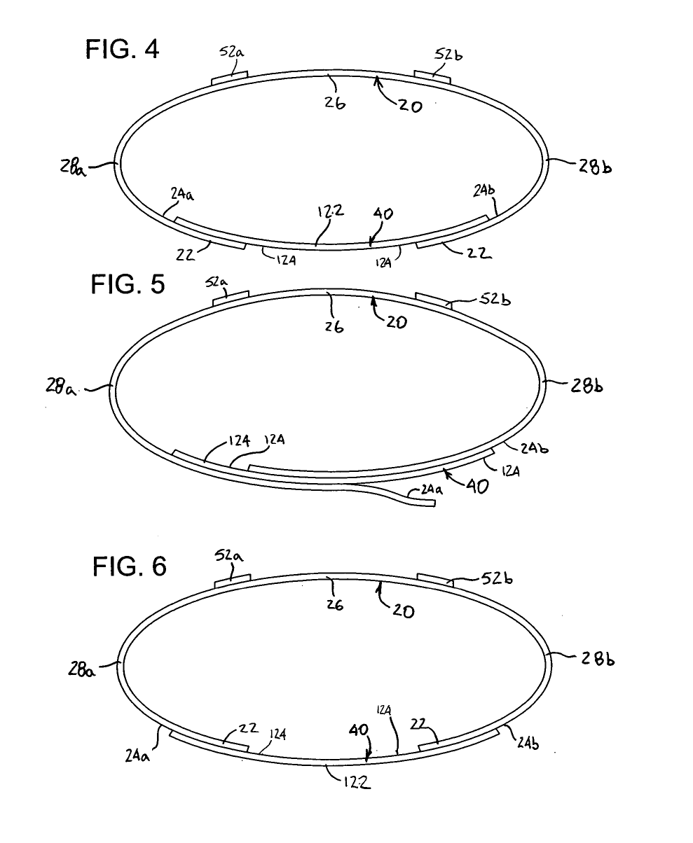

FIGS. 4-6 demonstrate several alternative belt member configurations. FIGS. 4 and 6 are related and differ only with respect to the sides of anterior portions 22a and 22b, and the sides of anterior suspension portion 122 to which the first and second parts of the fasteners are located. FIG. 5 provides a solution for extremely obese persons while at the same time permitting use by the less obese or those with smaller body diameters at the point of belt location.

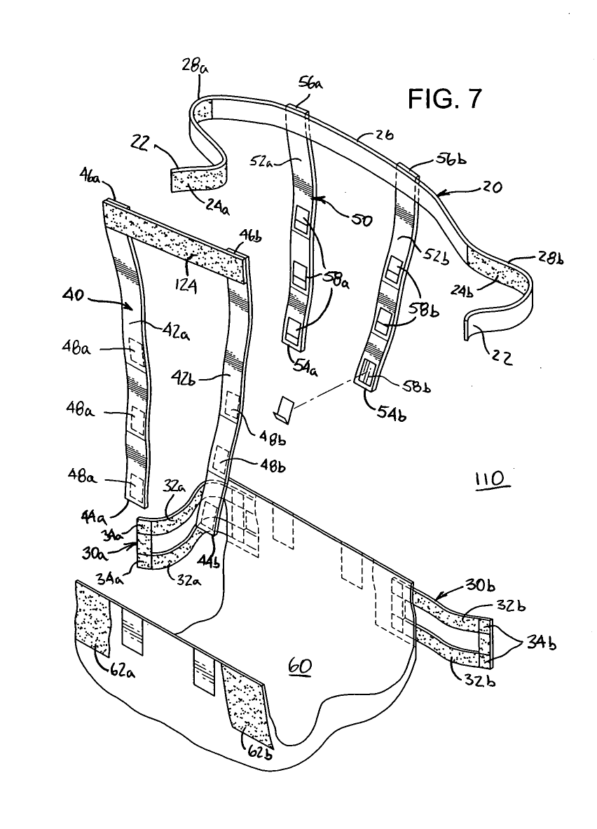

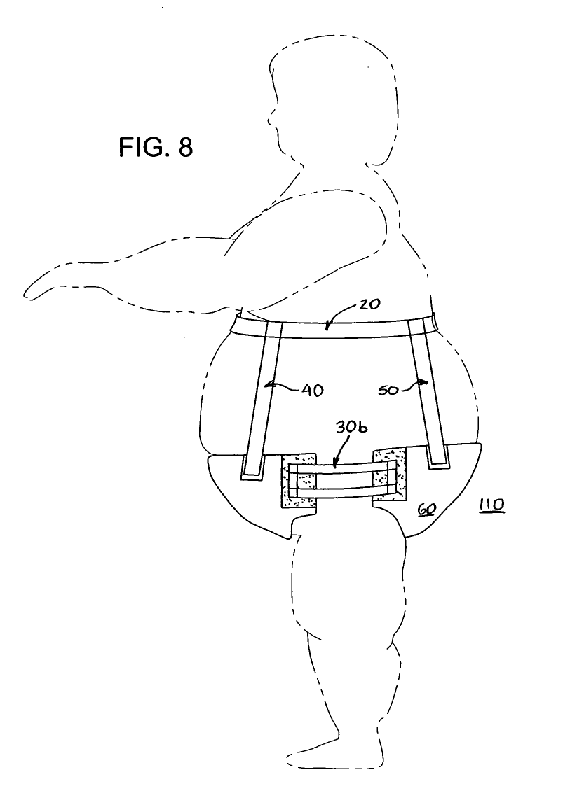

An alternative undergarment 110 to that shown in FIG. 1 is best illustrated in FIGS. 7 and 8. In this embodiment, the general structure of the anterior belt portion 22 (also in these embodiments referred to as anterior suspension portion 122), anterior suspension member 40, and posterior suspension member 50 remain substantially the same, as does the general nature and configuration of absorbent panel 60. However, lateral connection members 30a and 30b are employed to provide additional support and fitment of absorbent panel 60. As best shown in these Figures, lateral connection members 30a and 30b comprise lateral straps 32a and 32b, each of which has respective first-ends 34a and 34b. Respective first ends 34a and 34b cooperatively engage corresponding attachment points 62a and 62b on absorbent panel 60. As with respect to the first embodiment, first ends 34a and 34b may be attached to absorbent panel 60 in any conventional manner; however, removable attachment means such as the use of hook-and-loop fasteners, temporary adhesives and the like may be beneficially used. Thus, when a user wears undergarment 110, compression around the user's hip may be enhanced through the engagement of first ends 34a and 34b with attachment points 62a and 62b, as is best shown in FIG. 8. Again, it should be noted that only at least one end of lateral straps 32a and 32b should be removably engagable with absorbent panel 60; the second ends of lateral straps 32a and 32b may also, but need not, be removable. Fitment advantages can be realized by having both ends removably attached to absorbent panel 60; however, manufacturing and costing considerations may create additional advantages for embodiments wherein only a single end is removable.

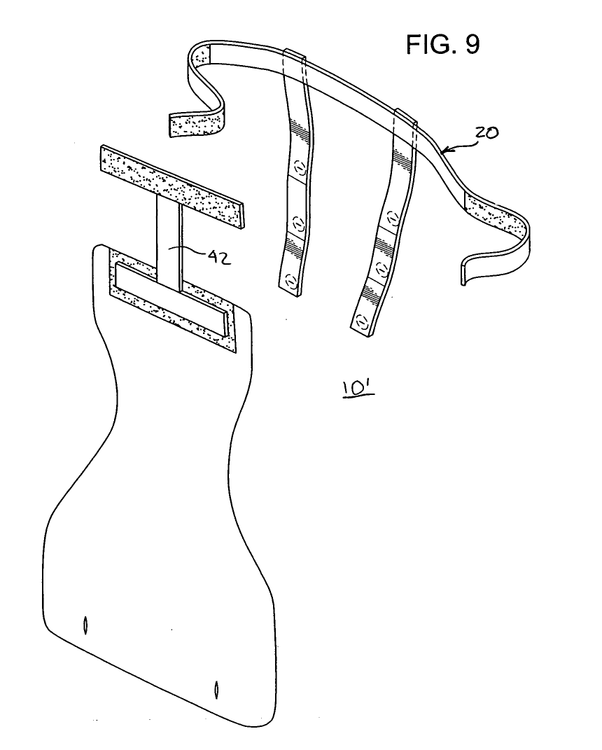

FIG. 9 is provided to illustrate that the anterior or posterior suspension member may comprise a single vertical strap; in this Fig., strap 42′ illustrates this option. Such a configuration may find usefulness in situations wherein the user has medical reasons that require a midline mode of attachment, e.g., a recent surgical closure. As with all prior embodiments, various modes for attachment are shown to illustrate the diversity and not necessarily a preferred mode of implementation.

1. An incontinence undergarment that during use comprises:
an absorbent panel having an outside surface, an inside surface, an anterior portion, a mid portion and a posterior portion wherein the panel has a fluid absorption value;
at least one anterior vertical suspension member extending from the front portion of the panel in a direction substantially opposite from the mid section;
at least one posterior vertical suspension member extending from the rear portion of the panel in a direction substantially opposite from the mid section; and
a belt member sized to at least partially surround a torso portion of a user,
wherein the at least one anterior vertical suspension member and the at least one posterior vertical suspension member extend from the belt member.

2. The undergarment of claim 1 wherein the belt member comprises two portions, an anterior portion extending from the at least one anterior vertical suspension member and a posterior portion extending from the at least one posterior vertical suspension member.

3. The undergarment of claim 1, further comprising at least one lateral connection member to link the anterior portion of the panel to the posterior portion of the panel.

4. The undergarment of claim 3 wherein the at least one lateral connection member has a fluid absorption value less than the fluid absorption value of the panel.

5. The undergarment of claim 1 wherein one of the at least one anterior or the at least one posterior vertical suspension member is a strap having a length and a width.

6. The undergarment of claim 1 wherein at least one of the at least one anterior or the at least one posterior vertical suspension member is adjustable in length relative to the panel.

7. The undergarment of claim 6 wherein the length adjustment comprises looping a portion of a vertical suspension member through an orifice associated with the panel and attaching a distal end of the suspension member to a portion thereof.

8. The undergarment of claim 1 wherein the at least one anterior vertical suspension member is removable from the belt member.

9. The undergarment of claim 2 wherein the at least one anterior vertical suspension member and the anterior portion of the belt is removable from the posterior portion of the belt member.

10. The undergarment of claim 1 wherein at least one of the at least one anterior or the at least one posterior vertical suspension member has a fluid absorption value less than the fluid absorption value of the panel.

11. A garter for use with an absorbent panel having an outside surface, an inside surface, an anterior portion, a mid portion and a posterior portion, the garter comprising when worn by a user:
a belt member sized to surround a torso portion of a user, the belt member having an anterior segment, a posterior segment and two connecting segments joining the anterior and posterior segments;
at least one anterior vertical suspension member extending a sufficient distance from the anterior segment of the belt member to terminate proximate to or above the pelvis of the user; and
at least one posterior vertical suspension member extending a sufficient distance from the posterior segment of the belt member to terminate proximate to or above the pelvis of the user.

12. The garter of claim 11 wherein the belt member comprises two portions, one portion extending from the at least one anterior vertical suspension member and the other portion extending from the at least one posterior vertical suspension member.

13. The garter of claim 11 wherein one of the at least one anterior or the at least one posterior vertical suspension member is a strap having a length and a width.

14. The garter of claim 11 one of the at least one anterior or the at least one posterior vertical suspension member is elastic.

15. The garter of claim 11 wherein at least one of the at least one anterior or the at least one posterior vertical suspension member is adjustable in length relative to the panel.

16. The garter of claim 11 wherein each at least one anterior and at least one posterior vertical suspension member has a distal end for connection with an absorbent panel, and wherein the garter further comprises a first part of a two part mechanical fastener for engaging the second part of the two part mechanical fastener located on the absorbent panel.

17. The garter of claim 11 wherein the at least one anterior vertical suspension member is removable from the belt member.

18. The garter of claim 11 wherein the at least one anterior vertical suspension member and the anterior segment of the belt member is removable from the belt member.
